(19) United States
(12) Patent Application Publication
(10) Pub. No.: US 2026/0021031 A1
(43) Pub. Date: Jan. 22, 2026

(54) CLEANSING AND TREATMENT SOLUTION FOR HUMANS

(71) Applicant: Moxikom, LLC, Oldsmar, FL (US)

(72) Inventors: Kimberly Moody, Oldsmar, FL (US); Bridget Conzemius, Oldsmar, FL (US)

(73) Assignee: Moxikom, LLC, Oldsmar, FL (US)

(21) Appl. No.: 18/777,680

(22) Filed: Jul. 19, 2024

(51) Int. Cl. A61K 8/92; A61K 8/19; A61Q 17/00; A61Q 19/10

(52) U.S. Cl. CPC A61K 8/922; A61K 8/19; A61Q 17/005; A61Q 19/10

(57) ABSTRACT

The present inventive concept relates to a formula for vaginal and anal cleansing and bacterial and microbial treatment of the same. The formula consists of a combination of Nano or Colloidal Siver, Melaleuca alternifolia which is Tea Tree and either distilled water or purified water. The formula is preferably applied through an applicator bottle or douche bottle.

### STATEMENT REGARDING FEDERALLY SPONSORED RESEARCH OR DEVELOPMENT

Not applicable.

### COPYRIGHT NOTICE

A portion of this disclosure contains material which is subject to copyright protection. The copyright owner has no objection to the photocopy reproduction by anyone of the patent document or the patent disclosure in exactly the form it appears in the Patent and Trademark Office patent file or records, but otherwise reserves all copyright rights whatsoever. 37 C.F.R 1.71(d).

### BACKGROUND OF THE INVENTIVE CONCEPT

### 1. Field of the Invention

The present inventive concept relates to a Nano or Colloidal Silver and Tea tree based solution, and more particularly to a Nano or Colloidal Silver and Tea tree based solution for vaginal and anal cleansing and treatment.

### 2. Description of the Related Art

It is well known that preparations using silver have been commonly used for germicide and antibiotic purposes. The use of silver preparations has been known to inhibit the growth of bacteria, viruses, and other unwanted organisms, as well as for eliminating such existing bacteria, viruses and other unwanted organisms. Further, it has also been known that solutions using silver have been shown to provide anti-inflammatory effects.

WO2006074117A2 by Holladay et al., discusses compositions containing silver nanoparticles that manifests significant antimicrobial properties. Specifically, this document discusses the use of silver particles in water at a level of about 5-40 ppm, which is used for treating infections and as disinfectants. However, this document does not disclose a solution that can be used in an applicator type container, and the use of silver alone is limited in its effect for cleansing and treatment.

WO2022146244 by Demirezen discusses a disinfectant composition used for personal hygiene, which includes herbal synthesized silver nanoparticles and water. However, this composition also includes other components such as myrrh tree plant extract, highbush cranberry leaf extract, sumac leaf extract, juniper fruit extract, jasmine and/or daisy flower extract, apple and sunflower face pomace. Further, this document does not address using its disinfectant composition for vaginal or anal cleansing.

Hence, there is a need for a specifically designed solution for providing an effective vaginal and anal cleansing and treatment.

There is also a need for a specifically designed solution for that has highly effective antimicrobial properties.

There is also a need for a specifically designed solution having a high antibiotic potency.

There is also a need for a specifically designed solution that has highly effective antimicrobial properties and high antibiotic potency, which can be stored in and administered from a douche or other type squeezable container for cleansing a vaginal and anal area.

### SUMMARY OF THE INVENTIVE CONCEPT

The present general inventive concept. More particularly, but not exclusively, the present inventive concept relates to a Nano or Colloidal Silver and Tea tree based solution, and more particularly to a Nano or Colloidal Silver and Tea tree based solution for vaginal and anal cleansing and treatment.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a cleansing or bacterial and microbial eradicating treatment formula, consisting of a mixture of the following ingredients: 10-30 parts-per-million PPM (or 200-400 Micrograms) of Nano or Colloidal Silver; 0.50%- 1.5% of Melaleuca alternifolia (Tea tree oil); and 113.39808 grams of distilled water or purified water. It should be known that greater or less amounts of the formula can be made by adding or decreasing each of the separate ingredients in equal proportions.

The foregoing and/or other features and utilities of the present general inventive concept may also be achieved by providing a method of making a cleansing or bacterial and microbial eradicating treatment formula, comprising the steps of: adding 10-30 parts-per-million PPM (or 200-400 Micrograms) of Nano or Colloidal Silver to a four-ounce bottle; adding 0.50%-1.5% of Melaleuca alternifolia (Tea tree oil) to the four-ounce bottle; adding 113.39808 grams of distilled water or purified water to the four-ounce bottle; and shaking the mixture in the four-ounce bottle until fully combined into a solution. Greater or less amounts of the formula can be made by adding or decreasing each of the separate ingredients in equal proportions.

Additional features and utilities of the present general inventive concept will be set forth in part in the description which follows and, in part, will be obvious from the description, or may be learned by practice of the general inventive concept.

### DETAILED DESCRIPTION OF THE PREFERRED EMBODIMENTS

The following description is provided to enable those skilled in the art to make and use the herein described invention and to set forth the best modes contemplated by the inventors of carrying out their invention. Various modifications, however, will remain readily apparent to those skilled in the art, since the general principles of the present inventive concept have been defined herein specifically to provide an improved colloidal silver product with significant effects in providing antimicrobial properties and high antibiotic potency for cleansing and treating the vaginal and anal areas of a human body.

The present inventive concept represents a novel approach to cleansing the vaginal and anal regions of a human body for proper hygiene and to eradicating bacteria, microbials and other harmful microorganisms from the vaginal and anal regions of a human body.

### Preferred Embodiments

Non-limiting preferred embodiments are presented by the following: A composition comprising a formula of nano or colloidal silver particles together with Melaleuca alternifolia (or tea tree) and distilled or purified water. More specifically, the present inventive concept comprises, in specific proportions to fill a four ounce bottle or other type container, a formula consisting of a range of 10-30 parts-per-million PPM (or 200-400 Micrograms) of Nano or Colloidal Silver liquid to 0.50%- 1.5% (of a four ounce bottle) of Melaleuca alternifolia (tea tree oil) to 113.39808 grams of distilled or purified water. This range of ingredients will result in the formula that will provide the desired results for providing the cleansing and treatment solution for vaginal and anal regions of a human body, according to the present inventive concept. Greater or less amounts of the resulting formula can be made by increasing or decreasing each of the separate ingredients in proportions to result in more or less of the same present inventive formula, without departing from the spirit and scope of the overall inventive concept.

A recent study published by “The Journal of Plant Medicines” (by Case Adams, Naturopath) has found that “the antibiotic potential of two natural antiseptics, Tea tree oil and Silver, can be significantly boosted and sustained using liposomal encapsulation.” These researchers found that the liposomal encapsulation of tea tree oil, silver ions separately and a combination of the two significantly reduced the viability of three microorganisms (Pseudomonas aeruginosa, Staphylococcus aureus and Candida albicans) known for their ability to evade many pharmaceutical antibiotics. Liposomal encapsulation is the blending of a dissolved solution together with a lipid carrier, which when done correctly, a certain amount of the dissolved solution will become absorbed into the lipid membranes, essentially trapping it inside a fatty acid cell membrane. Moreover, in a related study regarding Tea tree oil, it has been found that Tea tree oil produces little or no antibiotic resistance. Research has also indicated that most lethal bacterial agents are becoming increasing antibiotic resistant, making these lethal bacteria increasingly untreatable by antibiotics. However, this was not the case with Tea tree oil.

Another study by the Society for General Microbiology, published in Scientific Daily, Apr. 6, 2009, has indicated that “mixing tea tree oil and silver or putting them in liposomes, greatly increases their antimicrobial activity and may minimize any side effects.” Also indicated is that “although both tea tree oil and silver, as silver nitrate, are effective against a range of micro-organisms, when low concentrations of the two agents were combined, their antimicrobial activity increases.”

According to the International Wound Journal, Apr. 14, 2017, although traditional wound treatment regimes can no longer cope with the complications posed by antibiotic-resistant strains, pre-antibiotic compounds, including heavy metal ions and essential oils, pre-antibiotic compounds, including heavy metal ions and essential oils have been re-investigated for their potential use as effective antimicrobial agents. It has been concluded that the ‘versatility of . . . agents such as TTO (tea tree oil) and Ag+ against a wide range of different microorganisms because of their multiple target sites impedes the development of resistance and might be useful in improving the current wound treatment strategies. Based on the current findings, which showed the efficacy and beneficial therapeutic properties of both Ag⁺ and TTO, the potential advantages of using these agents in wound treatment regimes should be explored further.”

The inventors herein have determined that a formula specifically consisting of proportions (to fill a four ounce bottle or other type container) in the range of 10-30 parts-per-million PPM (or 200-400 Micrograms) of Nano or Colloidal Silver liquid to 0.50%- 1.5% (of a four ounce bottle) of Melaleuca alternifolia (tea tree oil) to 113.39808 grams of distilled or purified water, mixed together as a solution, provides a highly effective cleansing solution as both vaginal applications and anal applications. It is noted that larger or smaller amounts of the inventive formula can be made by increasing or decreasing the amounts of each of the separate ingredients in equal proportions, without departing from the spirit and scope of the overall inventive concept. In addition, the inventors have determined that this formula also provides an anti-microbial treatment for vaginal applications and anal applications.

This formula consisting of proportions, to fill a four ounce bottle or other type container, of 10-30 parts-per-million PPM (or 200-400 Micrograms) of Nano or Colloidal Silver liquid to 0.50%- 1.5% (of a four ounce bottle) of Melaleuca alternifolia (tea tree oil) to 113.39808 grams of distilled or purified water, mixed together as a solution, is found to be easily applied by placing the formula in a specifically designed douche bottle (a four ounce bottle for the above stated proportions of ingredients) or applicator type container and then executing the proper operations of inserting the douche bottle or applicator type container into the vaginal or anal areas for a thorough cleansing thereof while allowing the formula to flush back out of the vaginal or anal areas naturally. It is to be noted that the proportions of each of the ingredients can be adjusted proportionally to result in a greater amount of the overall formula or a lesser amount of the overall formula, without departing from the spirit and scope of the overall present inventive concept.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A cleansing or bacterial and microbial eradicating treatment formula, consisting of a mixture of the following ingredients:
10-30 parts-per-million PPM (or 200-400 Micrograms) of Nano or Colloidal Silver;
0.50%- 1.5% of Melaleuca alternifolia (Tea tree oil); and
113.39808 grams of distilled water or purified water.

2. A method of making a cleansing or bacterial and microbial eradicating treatment formula, comprising the steps of:
adding 10-30 parts-per-million PPM (or 200-400 Micrograms) of Nano or Colloidal Silver to a four-ounce bottle;
adding 0.50%- 1.5% of four ounces of Melaleuca alternifolia (Tea tree oil) to the four-ounce bottle;
adding 113.39808 grams of distilled water or purified water to the four-ounce bottle; and
shaking the mixture in the four-ounce bottle until fully combined into a solution.
